(19) United States
(12) Patent Application Publication
(10) Pub. No.: US 2025/0242175 A1
(43) Pub. Date: Jul. 31, 2025

(54) PREDICTING INTERNAL ANATOMICAL DEFORMATION FROM EXTERNAL BIOLOGICAL SIGNALS

(71) Applicant: Siemens Healthineers International AG, Steinhausen (CH)

(72) Inventors: Supratik BOSE, San Ramon, CA (US); Pascal PAYSAN, Basel (CH); Ricky R. SAVJANI, Los Angeles, CA (US)

(73) Assignee: Siemens Healthineers International AG, Steinhausen (CH)

(21) Appl. No.: 18/424,347

(22) Filed: Jan. 26, 2024

(51) Int. Cl. A61N 5/10; G16H 40/67; G16H 50/50

(52) U.S. Cl. CPC A61N 5/1037; A61N 5/1039; G16H 40/67; G16H 50/50

(57) ABSTRACT

Provided herein are methods and systems to train and execute a motion model that uses artificial intelligence methodologies to learn and predict location of a patient's internal structures. A method comprises receiving, via an electronic sensor, biological signal data of a patient; receiving a medical image of the patient depicting a planning target volume and at least one organ at risk of the patient, wherein the medical image received corresponds to the patient in a pre-treatment condition; executing an artificial intelligence model using the biological signal data and the medical image to predict deformation data for at least one of the at least one organ at risk or the planning target volume of the patient, wherein the artificial intelligence model is trained in accordance with a training dataset comprising a set of participants, their corresponding respiratory data, their medical images, and their corresponding deformation data; and outputting the deformation data.

### TECHNICAL FIELD

This application relates generally to using artificial intelligence modeling techniques to model and predict patient attributes.

### BACKGROUND

One of the major challenges in image-guided radiation therapy (IGRT) is addressing various types of patient motions. The motion can be both periodic/cyclical motion (e.g., components of respiratory and cardiac motion) as well as aperiodic/irregular motion (e.g., gastrointestinal events including peristalsis, swallowing, and the passage of gas bubbles, muscle relaxation in breath-hold, and body and limb movement).

IGRT attempts to mitigate the effects of motion in many ways, but there are deficiencies with conventional IGRT techniques. For instance, the imaging apparatus of the radiotherapy machine may not produce images during the radiation therapy of a patient. This may be due to the existence of radiation, which can impair the imaging apparatus. Accordingly, there may be no real-time three-dimensional (3D) volumetric depiction of patient anatomy/motion during treatment. Although there are imaging techniques attempting to resolve motion with respect to the respiratory or cardiac cycle, conventional imaging devices, and systems do not provide real-time resolved 3D information about the patient at every time instance.

### SUMMARY

For the aforementioned reasons, there is a desire for a system that can rapidly and accurately analyze patient information and provide a projected location of a patient's internal structures. For instance, there is a desire to generate/predict a moving image of the patient's internal organs while being treated without the need to use a real-time or near-real-time image of the patient captured by imaging apparatus. Using the methods and systems discussed herein, a computer model (e.g., artificial intelligence (AI) model) can ingest biological signals and analyze patient movements accordingly. Using the methods and systems discussed herein, a processor can use deep learning to train AI models to generate a synthetic medical image.

Applications of these computer models can be in the field of real-time (or near-real-time) tissue tracking during radiation beam delivery, real-time (or near-real-time) motion visualization, retrospective and/or real-time (or near-real-time) delivered dose calculation, organ/segmentation (e.g., planning target volume (PTV) or gross tumor volume (GTV)), specific dose tracking, outcome prediction, image reconstruction, and the like.

During external beam radiotherapy sessions, medical professionals benefit from knowing the position and boundary of a PTV/GTV (e.g., tumor) being treated. Such knowledge allows accurate delivery of radiation to the tumor during treatment and results in the sparing of normal tissue of organs at risk (OARs) from radiation and associated toxicity. However, even when the patient lies still on the radiotherapy couch, their internal anatomy may move/deform in both periodic and aperiodic fashion, as discussed herein. Based on the anatomical site, some tumors may move more or in different shapes/manner compared to others.

The uncertainty of the tumor position and deformation due to internal movement may be accounted for (at least to a certain extent) in the form of a margin around tumor volume during treatment planning. For instance, a bigger tumor volume may be assumed when planning radiation, such that the deformation is also accounted for. However, if there is a mechanism available to know the position of the target tumor during a radiotherapy session with more certainty and accuracy, various measures may be possible to improve the patient's treatment (e.g., reducing margin in treatment planning, stopping radiation (“beam hold”) when the tumor moves out of the path of radiation more than the planned margin, modifying the path of radiation (Multi-Leaf Collimator aperture dynamically tracking a moving tumor), and/or compensating for dose deviation in follow-up fractions based on a review of modeled target movement (as a function of external biological signal).

Obtaining continuous (“live”) accurate volumetric three-dimensional (3D) images of internal anatomy (both the tumor and surrounding OARs) during radiotherapy treatment sessions is a technically challenging task. Strategies for obtaining the “live” position of the tumor may include the following.

For full 3D imaging using non-ionizing method, recent advances in the combination of Magnetic Resonance Imaging (MRI) with Linear Accelerator (LINAC), namely MR-LINAC, attempt to provide continuous 3D imaging during radiation. However, utilizing this method has proven to be costly.

For 2D monoscopic, stereoscopic, or tomosynthesis-like imaging using X-ray, most conventional methods acquire intermittent X-ray projections with X-ray sources either mounted and moving with a gantry or X-ray sources (typically 2) mounted at fixed positions in the treatment room. However, fast and accurate detection and tracking of targets in 2D X-ray projections is also a challenging task due to the lack of contrast caused by factors like scatter, occlusion by other organs (based on view angle), and the like. Despite the reduced contrast, various attempts are made to track the projection of the target tumor in the X-ray projections and then obtain the 3D position using projection geometry. Also, such X-ray projections, acquired over a short duration, are combined (tomosynthesis reconstruction) to produce pseudo-3D-like imaging of internal anatomy in which one attempts to track the target tumor, though with a delay sometimes.

Using the methods and systems discussed herein, a processor can use various modeling techniques to model target PTVs that are in an anatomical region where motion is periodic and correlated with breathing or cardiac motion (e.g., solid tumors in lungs or those close to the diaphragm). These modeling techniques attempt to predict the movement of the target and surrounding anatomy as a function of an external biological signal (sometimes referred to as a surrogate signal). Non-limiting examples of an external biological may include a breathing signal captured via a chest belt, spirometer, bellows, a breathing signal captured using imaging apparatus, kilo-voltage imaging (kv imaging), and/or cardiac signal captured via echocardiogram.

In some embodiments, to generate such a model, there may be a need for simultaneous (or contemporaneous) acquisition and reconstruction of an image or volume of the internal anatomy along with concurrent/correlating capture of the external biological signal. Then, one or more processors can be programmed to generate a model of the deformation of internal anatomy as a function of change in the external biological signal. As a result, if such a relationship is consistent during a radiation therapy treatment session, the model can accurately deform the pre-treatment image or volume of internal anatomy as a function of the external biological signal observed during treatment and predict the internal anatomy and position of the target during treatment.

In an embodiment, a method comprises receiving, by a processor via an electronic sensor, biological signal data of a patient; receiving, by the processor, a medical image of the patient depicting a planning target volume and at least one organ at risk of the patient, wherein the medical image received corresponds to the patient in a pre-treatment condition; executing, by the processor, an artificial intelligence model using the biological signal data and the medical image to predict deformation data for at least one of the at least one organ at risk or the planning target volume of the patient, wherein the artificial intelligence model is trained in accordance with a training dataset comprising a set of participants, their corresponding respiratory data, their medical images, and their corresponding deformation data; and outputting, by the processor, the deformation data.

The medical image may be a free-breathing image of the patient.

The artificial intelligence model may be configured to use a moving or fixed portion of the planning target volume or the at least one organ at risk within the medical image to predict the deformation data.

The biological signal data may be received from the electronic sensor is at least one of a chest position, chest movement, or respiratory cycle data of the patient.

The deformation data may correspond to a movement, change in shape, or a position of at least one of the planning target volume or the at least one organ at risk of the patient.

The method may further comprise adjusting, by the processor, at least one attribute of a radiotherapy machine in accordance with the deformation data.

The electronic sensor may be a wearable respiratory sensor or an optical respiratory sensor.

Outputting the deformation data may correspond to a simulated medical image depicting an anatomical region of the patient.

Outputting the deformation data corresponds to transmitting the deformation data to a dose calculation software solution or a tissue tracking software solution.

In another embodiment, a computer system comprises a server comprising a processor and a non-transitory computer-readable medium containing instructions that when executed by the processor causes the processor to perform operations comprising: receiving, via an electronic sensor, biological signal data of a patient; receiving a medical image of the patient depicting a planning target volume and at least one organ at risk of the patient, wherein the medical image received corresponds to the patient in a pre-treatment condition; executing an artificial intelligence model using the biological signal data and the medical image to predict deformation data for at least one of the at least one organ at risk or the planning target volume of the patient, wherein the artificial intelligence model is trained in accordance with a training dataset comprising a set of participants, their corresponding respiratory data, their medical images, and their corresponding deformation data; and outputting the deformation data.

The medical image may be a free-breathing image of the patient.

The artificial intelligence model may be configured to use a moving or fixed portion of the planning target volume or the at least one organ at risk within the medical image to predict the deformation data.

The biological signal data received from the electronic sensor may be at least one of a chest position, chest movement, or respiratory cycle data of the patient.

The deformation data may correspond to a movement, change in shape, or a position of at least one of the planning target volume or the at least one organ at risk of the patient.

The instructions may further cause the processor to adjust at least one attribute of a radiotherapy machine in accordance with the deformation data.

The electronic sensor may be a wearable respiratory sensor or an optical respiratory sensor.

Outputting the deformation data may correspond to a simulated medical image depicting an anatomical region of the patient.

Outputting the deformation data may correspond to transmitting the deformation data to a dose calculation software solution or a tissue tracking software solution.

In another embodiment, a system comprises a radiotherapy machine; a data repository configured to store an artificial intelligence model; a processor configured to receive, via an electronic sensor, biological signal data of a patient; receive a medical image of the patient depicting a planning target volume and at least one organ at risk of the patient, wherein the medical image received corresponds to the patient in a pre-treatment condition; execute the artificial intelligence model using the biological signal data and the medical image to predict deformation data for at least one of the at least one organ at risk or the planning target volume of the patient, wherein the artificial intelligence model is trained in accordance with a training dataset comprising a set of participants, their corresponding respiratory data, their medical images, and their corresponding deformation data; and output the deformation data.

The medical image may be a free-breathing image of the patient.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an artificial intelligence motion modeling system in accordance with an embodiment.

FIG. 2 illustrates a process flow diagram of an artificial intelligence motion modeling system, in accordance with an embodiment.

FIGS. 3-9 illustrate visual representations of training and implementing an AI model, in accordance with an embodiment.

FIG. 10 illustrates an example of a medical image generated in an artificial intelligence motion modeling system, in accordance with an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used, and/or other changes may be made without departing from the spirit or scope of the present disclosure. The illustrative embodiments described in the detailed description are not meant to be limiting of the subject matter presented.

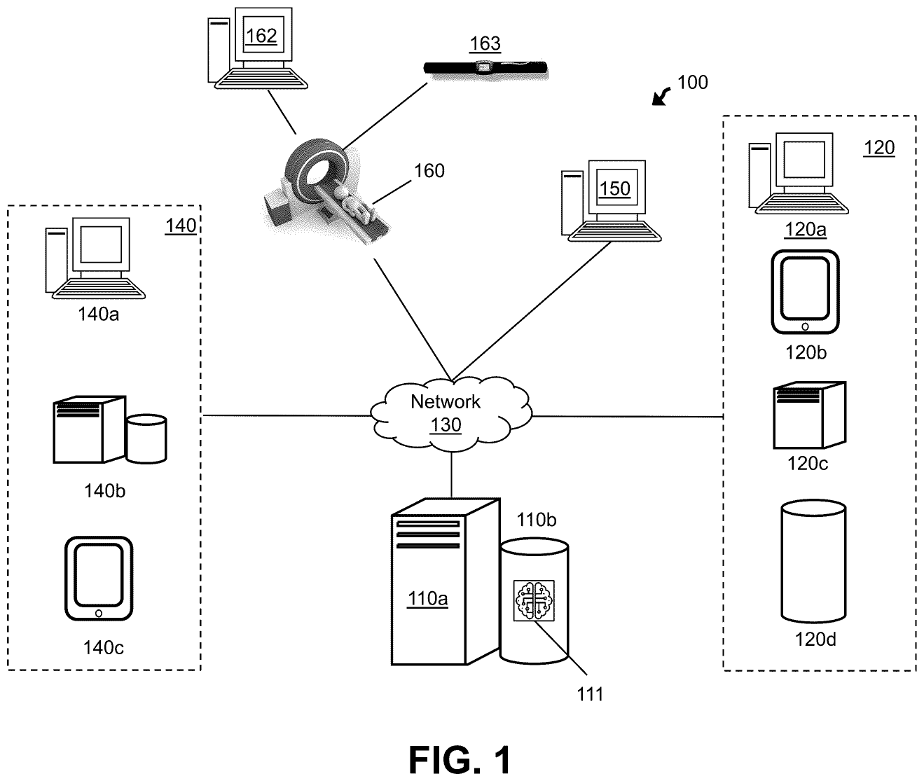

FIG. 1 illustrates components of a system 100 for an artificial intelligence motion modeling system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, and medical device 160, and medical device computer(s) 162. Various components depicted in FIG. 1 may belong to a radiotherapy clinic at which patients may receive radiotherapy treatment, in some cases via one or more radiotherapy machines located within the clinic (e.g., medical device 160).

The system 100 is not confined to the components described herein and may include additional or other components, not shown for brevity, which are to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), and EDGE (Enhanced Data for Global Evolution) network.

The analytics server 110a may generate and display an electronic platform configured to use various AI model 111 (including artificial intelligence and/or machine learning models) for receiving patient information and outputting the results of execution of the AI model 111. The electronic platform may include graphical user interfaces (GUI) displayed on each electronic data source 120, the end-user devices 140, the medical device 160, and/or the administrator computing device 150. An example of the electronic platform generated and hosted by the analytics server 110a may be a web-based application or a website configured to be displayed on different electronic devices, such as mobile devices, tablets, personal computers, and the like.

The information displayed by the electronic platform can include, for example, input elements to receive data associated with a patient being treated, synchronize one or more sensors, and display results of predictions produced by the AI model 111. For instance, the analytics server 110a may execute the AI model 111 (e.g., machine learning models trained to generate predicted tumor locations and/or breathing patterns for a patient being treated via the medical device 160). The analytics server 110a may then display the results for a medical professional and/or directly revise one or more operational attributes of the medical device 160. In some embodiments, the medical device 160 can be a diagnostic imaging device or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various electronic data sources that contain, retrieve, and/or access data associated with a medical device 160, such as operational information associated with previously performed radiotherapy treatments (e.g., electronic log files or electronic configuration files), data associated with previously monitored patients (e.g., breathing patterns, tumor location, deformation information) or participants in a study to train the AI models discussed herein. For instance, the analytics server 110a may use the clinic computer 120a, medical professional device 120b, server 120c (associated with a physician and/or clinic), and database 120d (associated with the physician and/or the clinic) to retrieve/receive data associated with the medical device 160. The analytics server 110a may retrieve the data from the end-user devices 120, generate a training dataset, and train the AI models 111. The analytics server 110a may execute various algorithms to translate raw data received/retrieved from the electronic data sources 120 into machine-readable objects that can be stored and processed by other analytical processes as described herein.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, or server computer. In operation, various users may use end-user devices 140 to access the GUI operationally managed by the analytics server 110a or otherwise the results of the execution of the AI model 111. Specifically, the end-user devices 140 may include clinic computer 140a, clinic server 140b, and a medical professional device 140c. Even though referred to herein as “end-user” devices, these devices may not always be operated by end-users. For instance, the clinic server 140b may not be directly used by an end user. However, the results stored on the clinic server 140b may be used to populate various GUIs accessed by an end user via the medical professional device 140c.

The administrator computing device 150 may represent a computing device operated by a system administrator. The administrator computing device 150 may be configured to display radiotherapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the AI model 111 that are maintained by the analytics server 110a.

The medical device 160 may also include one or more sensors configured to monitor the patient being treated. That is, the medical device 160 and/or the analytics server 110a may be communicating with various sensors that can monitor a patient's external biological signals. Non-limiting examples of the sensors may include 3D surfacing mechanisms and optical (or other) sensors configured to monitor the patient's movements (e.g., how the patient is moving and/or breathing). A non-limiting example of the sensors may include a respiratory sensor 163. The respiratory sensor 163 may be any sensor configured to monitor the patient's breathing. For instance, the respiratory sensor may be a strap configured to monitor the patient's chest position and movement, whereby a processor (e.g., internal to the respiratory sensor 163 or the analytics server 110a) can analyze to identify how the patient is breathing. Data received from the respiratory sensor 163 may also be referred to as the external biological signal or the surrogate signal.

The Al model 111 may be stored in the system database 110b. The AI model 111 may be trained using data received/retrieved from the electronic data sources 120 and may be executed using data received from the end-user devices, the medical device 160, and/or the sensor 163. In some embodiments, the AI model 111 may reside within a data repository local or specific to a clinic. In various embodiments, the AI model 111 may use one or more deep learning engines to generate predicted organ deformity for a patient being treated. For instance, the analytics server 110a may transmit patient attributes from the sensor 163 and execute the AI model 111 accordingly. The analytics server 110a may then display the results on one or more end-user devices 140. In some embodiments, the analytics server 110a may change one or more configurations of the medical device 160 based on the results predicted by the AI model 111.

It should be understood that any alternative and/or additional machine learning model(s) may be used to implement similar learning engines. The deep learning engines can include processing pathways that are trained during a training phase. Once trained, deep learning engines may be executed (e.g., by the analytics server 110a) to generate predicted patient attributes.

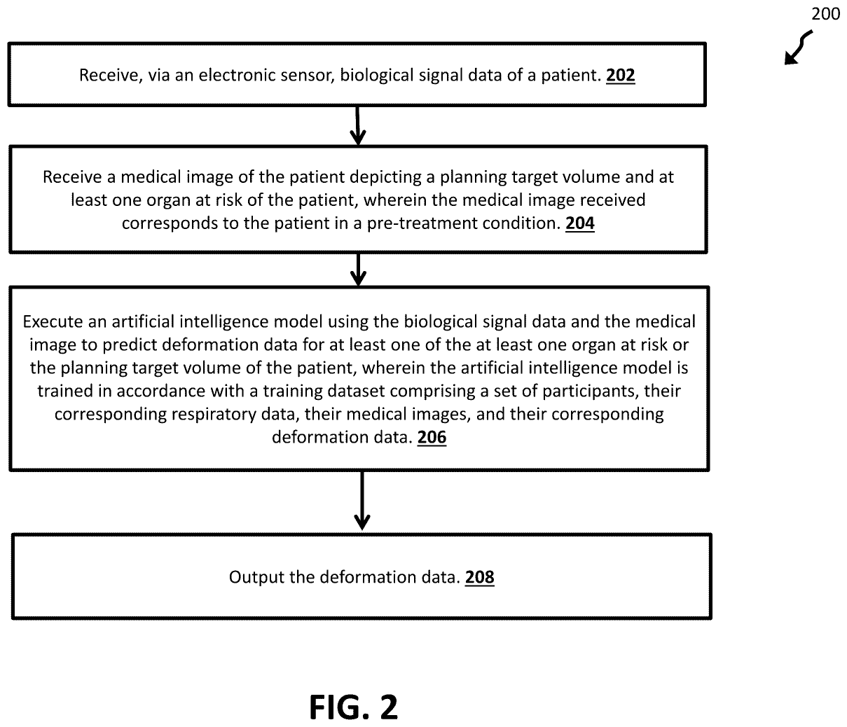

Referring to FIG. 2, depicted is an example data flow diagram 200 that shows how an Al model can be trained and executed to predict a patient attribute, in accordance with an embodiment. The method 200 may include steps 202-208. However, other embodiments may include additional or alternative steps or may omit one or more steps altogether. The method 200 is described as being executed by a server, such as the analytics server described in FIG. 1. However, one or more steps of method 200 may be executed by any number of computing devices operating in the distributed computing system described in FIG. 1. For instance, one or more computing devices may locally perform part, or all of the steps described in FIG. 2.

As discussed herein, patients usually experience internal organ movement during radiotherapy treatment. The movement may be caused by both periodic and aperiodic factors. In some embodiments, periodic movements are often due to respiration and cardiac activity, while aperiodic movements might arise from other factors like gas bubbles. These internal movements can be considered 3D movements (e.g., in multiple directions). In some cases, these internal movements often correspond to external indicators. That is, the internal organ movement of a patient may also manifest externally or vice versa. Using the methods and systems discussed herein (e.g., method 200), an AI model can be trained such that the external indicators (e.g., chest movement that is measured via a chest belt or an optical sensor) are ingested by the AI model, and internal movements are predicted.

One of the primary objectives of radiotherapy may be to accurately target the PTV with radiation while minimizing exposure to surrounding healthy tissue (e.g., OARs). Understanding and tracking a patient's internal movements can be used to predict the location of PTV(s) and OAR(s). In some embodiments, the model discussed herein can be implemented to create a continuous 3D representation of a patient's internal movements.

Using the methods and systems discussed herein, the analytics server can acquire both external biological signals and internal 3D imaging (in a timely manner such that they correspond to each other, for instance, simultaneously or near-simultaneously) prior to a patient's treatment. For instance, for each patient, one or more medical images depicting the internal structures can be collected alongside corresponding (e.g., paired) breathing signals or other surrogate signal data.

This process may involve capturing a 3D image of the patient paired with a breathing signal and then repeating this with another 3D image and a different breathing signal. During training, the AI model may uncover various patterns and correlations between these sets of 3D volumes and their corresponding external signals to identify movements, such as the diaphragm shifting or voxel displacement. Once such datasets are ingested by the AI model, the Al model may establish a relationship between changes in the external biological signal and internal voxel movements within the 3D volumes depicting each patient's internal structures. This forms the basis of a patient-specific model. The AI model may also establish a relationship between how the external signal changes vs how the internal organs move.

The AI model may predict how the entire (or parts of) the 3D volume changes in response to variations in the external signal. For example, the AI model may associate different types of volumes, such as free-breathing volumes or breath-hold volumes, with corresponding breathing signals, whether continuous or discrete.

In some embodiments, prior to the treatment, a 3D volume and its associated external signal can be recorded and transmitted to the AI model. As a result, the AI model may establish a relationship specific to the patient (though the AI model is trained using other patients' data). Subsequently, during the patient's treatment, only the breathing signal may be monitored, and the results can be ingested by the AI model, such that the internal movement of the patient is predicted.

Using the method 200 allows for predictions of internal volume changes based on the observed breathing patterns without directly imaging the patient during treatment. As a result, if a patient holds their breath at a certain level before and during treatment, the model can predict internal volume changes based on any differences in the breathing level.

### Training the Artificial Intelligence Model

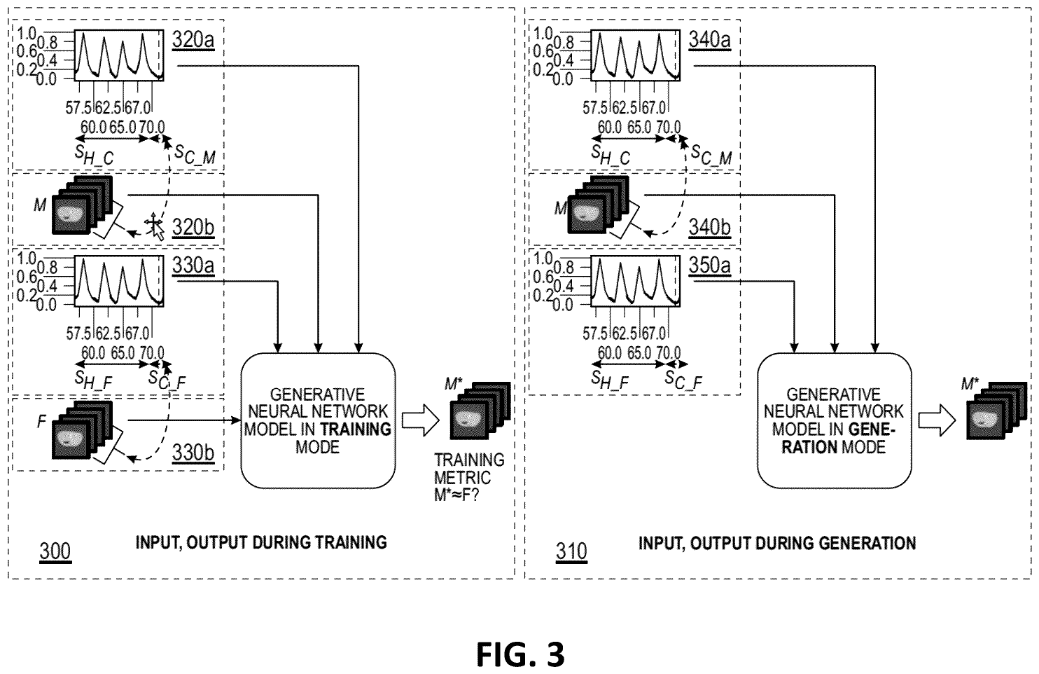

Before executing the AI model, the analytics server may first train the AI model and ensure its accuracy, as depicted in FIGS. 3-9.

FIGS. 3-9 describe training an AI model that, during prediction, may generate a deformed volume from a reference volume conditional on the change of a biological signal. In some embodiments, the biological signal is described as being associated with a patient's breathing. However, the AI model's use of a biological signal may not be limited to breath-related signals/surrogates. In some embodiments, any biological signal, such as an electrocardiogram, which accompanies a 3D volume reconstruction, e.g., cardiac CT, may be used. Therefore, no limitation is intended by any of the embodiments described herein.

FIG. 3 illustrates a generative neural network model (sometimes referred as the model or AI model for brevity) that can be trained to predict a deformed 3D volumetric CT image corresponding to a free-breathing signal (and signal history) from another 3D volumetric CT and its corresponding free-breathing signal (and signal history). The depicted AI model may use free breathing CT scans (e.g., 25 per patient) and corresponding surrogate breathing signals (e.g., one-dimensional) to AI model the deformation. The depicted AI model may also be generated using a population-based generative neural network model. That is, the generative neural network model discussed in FIG. 3 can be trained using data associated with a cohort of patients. However, the AI model may be personalized to a particular patient using their specific free-breathing signal and medical images.

The AI model may be implemented in two separate phases. The first phase (training phase 300) and the subsequent phase (generation phase 310). In the training phase 300, the analytics server may train the generative model, and in the generation phase 310, the analytics server may use test patients to use the AI model to generate predictions. The AI model may use the following inputs for training:

F: Thin slab of slices from a free-breathing CT scan referred to as a “Fixed scan”.

### Conditional Input:

M: Thin slab of slices from a free-breathing CT scan at the same anatomical region as that of F but acquired at a different breathing period, referred to as a “Moving scan.”

S_{M}:<S_{M_H}, S_{M_C}>1D: breathing surrogate signal consisting of signal history prior to the acquisition of the slices of M as well as current 1D breathing surrogate signal matching the slices of M.

S_{F}:<S_{F_H}, S_{F_C}>1D: breathing surrogate signal consisting of signal history prior to the acquisition of the slices of F as well as current 1D breathing surrogate signal matching the slices of F.

The AI model may have the following outputs:

M*: Thin slab of slices obtained by deforming slices of M, also referred to as Moved scan.

During the training of the AI model, a thin slab of slices of one scan (moving (M) scan) and corresponding 1D surrogate breathing signal SM (both history as well as the current signal matching slices of M) may be used as conditional input. The thin slab of slices of another scan (Fixed (F) scan) of the same anatomical region (but acquired at a different breathing period) may be used as input, while the 1D surrogate breathing signal SF (both history as well as the current signal matching slices of F) may also be used as conditional input.

During training, the AI model may analyze/model the deformation between F and M conditioned in light of the surrogate signals. The AI model may then produce a deformed version of M, namely M*. The mean square error between M* and F may be used as the error signal to train the AI model. Accordingly, during training, the pixel-wise mean square error between M* and F may be used to determine how well the training captures deformation from M to F as a function of change of 1D breathing surrogate signal from S_{M} to S_{F}.

The analytics server may implement the AI model using test patients. For instance, test patients can be used to train and improve the AI model's accuracy/precision. While using the trained AI model on a test patient in the generation mode, the test patient may be asked to breathe freely (e.g., a free-breathing scan M and corresponding surrogate signal SM (current and history) may be used as reference). Subsequently, when the test patient's data is ingested by the AI model along with any other surrogate breathing signal SF (current and history), the trained AI model may generate the deformed version of the scan M that matches the change in the surrogate from Sm to SF.

The analytics server may use a series (e.g., 16) of thin slabs of contiguous slices as 3D Moving and Fixed volume. While each free-breathing scan comprises around 150 to 225 slices, the thin slabs may be selected by moving a running partially overlapping window of a subset of the slices over the collection of all slices in a scan. “Moving” and “Fixed” slabs may be matching (same anatomical and Z position) sets of slices from a pair of free breathing scans of the same patient.

Selecting thin slab volume as input may result in a smaller memory footprint and GPU requirement. Moreover, it may also increase the number of data samples. Accordingly, the choice of using thin slab volume as input may also naturally support the simulation of 4DCT phase volume from the AI model that uses free-breathing CT volume as input.

As depicted in FIG. 3, while using the AI model in generation mode, given a free-breathing scan volume and corresponding free-breathing signal (current and history), the analytics server can provide any additional free-breathing signal (current and history) to obtain a deformed “free breathing” volume matching the current part of the additional free breathing signal. When a simulation of a breath-hold or 4DCT phase volume is desired, the current part of the second free breathing signal may correspond to a constant value for the whole scan, something that the AI model may not encounter in its free-breathing training data (especially when using a thick slab of many slices as training volume). However, if a thin slab volume is used in training, the value of the “current” part of the free-breathing signal is almost constant during the short duration of acquisition of the thin slab.

In a non-limiting example, if the thin slab constitutes of 16 slices in the current dataset, the acquisition time of this thin slab may be about 0.4 seconds, which matches the duration of one of 10 4DCT phase volumes that may be obtained from a 4-second breathing period. Therefore, by inputting such a second “almost constant” free-breathing signal, the AI model can obtain a simulated 4DCT phase volume for the whole scan.

The decision to use a thin slab and the decision to use breathing signal history along with the “current” breathing signal may be related and may work together to improve the results generated by the AI model. The “current” breathing signal matching the very short duration of acquisition of the thin slab may not contain long-term features of the breathing surrogate, including frequency, amplitude, drifts, etc. This may be needed for building a surrogate conditional model. The inclusion of historical breathing signals leading up to the acquisition may result in the inclusion of those features in modeling.

Using the method 200, the AI model may not need to ingest imaging data of the patient during treatment (or sometimes prior to the treatment) because the AI model is trained to predict changes on the pre-treatment imaging data based on the external signal alone.

The AI model discussed herein can also be configured to personalize its predictions for individual patients. However, in some embodiments, the AI model can be trained using a training dataset collected from a broad cohort of patients (e.g., previously treated patients). By analyzing motion patterns across a cohort of patients, the AI model can establish a robust correlation between biological signals and internal structural movements. As a result, the AI model may learn typical patient motions and create a more reliable model that reflects the general patient population's internal-external motion relationship, as opposed to a model that only applies to certain types of patients.

Because the AI model discussed herein is a deep-learning-based model, the AI model may not need many samples of a particular patient to predict that patient's internal movements. As discussed herein, the AI model discussed herein can build a statical relationship for a cohort of patients (and not particular patients as used in many conventional modeling paradigms). For instance, as depicted in FIG. 3, the AI model may ingest historical breathing signal 320a and the historical corresponding CT images 320b during the training phase 300. The AI model may also ingest a free-breathing signal for a patient (330a) and corresponding free-breathing CT images 330b. The CT images 330b may be used as ground truth. During the generation phase 310, the AI model may only need the historical signals (340a) and historical CT images 340b. The model may then ingest a free-breathing signal 350a and then predict (sometimes continuously) the CT images of the patient (e.g., during treatment). Therefore, unlike conventional models, the AI model discussed herein may only need historical images and signals and a few samples of free signals (free-breathing signals) to customize itself to a particular patient.

As discussed herein, the AI model may be trained using a diverse range of patients having different biometrics, such that the AI model's training is enhanced. For instance, each patient may contribute unique characteristics and biometric information, such as different body sizes (ranging from taller to shorter statures, including internal organs). This diversity can extend to their biological signal as well, such as breathing patterns, which can include slow, fast, shallow, or deep breathing. The AI model may function as a transformer, learning to interpret and correlate these varied biometrics with biological signals (e.g., breathing patterns). For instance, the AI model may process data from a patient with a larger chest volume and rapid breathing, as well as from another patient with a smaller volume and slower breathing. The AI model may learn to adapt to these differences, understanding how a particular biometric type combined with a specific biological signal may influence changes in the 3D volume during different breathing phases, such as inhaling to exhaling or transitioning between different points in the breathing cycle. This approach ensures that the AI model is not limited to patient-specific data but rather gains a comprehensive understanding across a broad spectrum of biometric and breathing variations from a cohort of patients.

For instance, as depicted in FIG. 3, the input images (referred to as images M) contribute to making the model patient-independent or patient-agnostic. As a result of ingesting much patient data within the cohort of patients used for training, the AI model may rely solely on the signals to predict internal motion without being tied to specific patient characteristics. Therefore, the AI model may be trained to analyze and understand the patient's anatomy. During training, the AI model may learn to identify the anatomical structures of different patients and may then adapt or deform the anatomical structures in response to biological signals. This process enables the AI model to accurately predict how different anatomical features would move or change in response to variations in biological signals. This paradigm allows the AI model to adapt to a wide range of patients and their unique physiological characteristics.

The AI model's capability may extend to accommodating a wide variety of patient scenarios, which include differences in lung volume and/or tumor characteristics. These variations can encompass large or small lung volumes, the presence of either a large or small tumor, and the tumor's proximity to the diaphragm. The AI model's learning process may not be limited to recognizing a single voxel; instead, it comprehends the spatial relationship and locality of each voxel in relation to surrounding anatomical structures, like the ribs and diaphragm.

This understanding enables the AI model to predict how groups of voxels move in response to changes in biological signals, such as breathing signals. The AI model may first consider the initial pretreatment volume (referred to as volume M) and may correlate it with changes in biological signals. The AI model may then learn to anticipate how the internal combination of these voxels will shift in response to alterations in biological signals.

The AI model's effectiveness or prediction accuracy may not be limited by the size or location of the tumor/PTV. Instead, the AI model may accurately capture movements for different tumor sizes and types, eliminating the need for highly specific adjustments for each treatment scenario. In some embodiments, the AI model may add an additional degree of freedom within the latent space in order to have a more patient-specific tailoring.

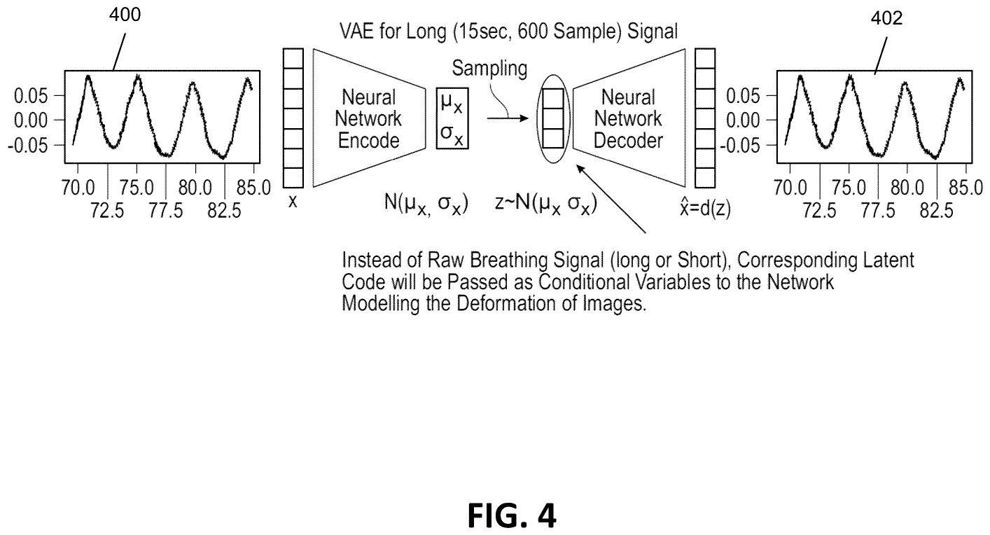

FIG. 4 illustrates how the AI model can analyze breathing signal history and current breathing signals. As described herein, the AI model may use both historical breathing signals prior to the acquisition of the thin slab of free-breathing CT slices as well as the current breathing signal matching the acquisition of the slices. As depicted, while the length of breathing signal history can be around 15 seconds, the length of the current breathing signal matching the duration of acquisition of a thin slab can be quite short, e.g., around 0.4 seconds for a slab of 16 slices. Due to such a large difference in the timescale, the AI model may not use the raw 15-second-long breathing history in the training phase. Instead, the AI model may first obtain a latent space representation of the 15-second-long one-dimensional breathing signal by first training a one-dimensional Variational Auto Encoder (VAE) model with various 15-second-long breathing samples.

The trained one-dimensional VAE itself can be a generative model. The trained one-dimensional VAE can be used to evaluate (e.g., when implemented in evaluation mode), where any 15-second-long breathing signal history associated with any scan is provided as the input. As a result, the VAE may produce the corresponding latent vector (of length 16 or 32 or as designed) generated by the encoder path of the trained VAE. For instance, the VAE may receive the breathing signal 400, transform the data into a latent space, and reproduce the breathing signal 402.

Accordingly, using the VAE and the latent vector, as described herein, can allow implicit capturing of short-term and long-term features of breathing history, which is depicted in FIG. 4. As depicted, the latent vector representation of the breathing history and the raw current breathing signal corresponding to the thin slab of slices together form the breathing surrogate.

In FIG. 4, the one-dimensional VAE is trained on 15-second-long breathing signals. Then, for any long breathing signal history to be used as a biological signal, the AI model may pass that signal to the trained ID VAE in evaluation mode and receive the generated latent code for use in the training of the motion model. During training of the main AI model to produce deformation vector, the trained 1-D VAE in FIG. 4 takes the “historical” (e.g., 15 sec long) part of the breathing signals S_{M} and S_{F}, namely S_{M_H} and S_{F_H} and convert them in latent variables S_{M_LV} and S_{F_LV} which are then used along with “current” part of the breathing signal, namely S_{M_C} and S_{F_C}.

It should be appreciated that FIG. 4 provides a non-limiting example of the VAE used by the AI model. Therefore, the lengths and durations used to illustrate the VAE are purely used to describe its functionality, and no limitation is intended. For instance, the description of FIG. 4 provides a 15-second-long breathing signal. However, other embodiments may involve other lengths of breathing signals and/or other biological signals.

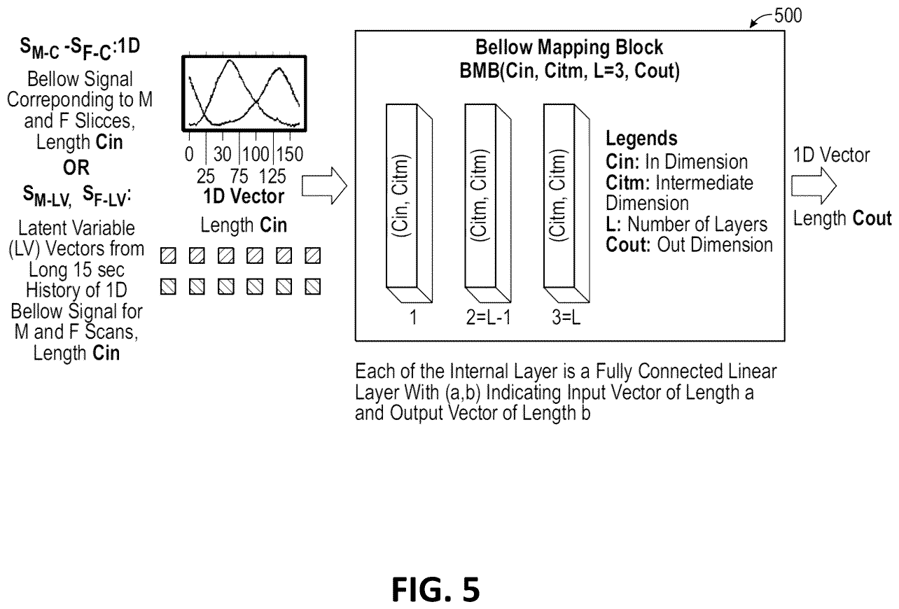

FIG. 5 describes a bellow mapping block. As used herein, a bellow mapping block may refer to a concatenation of fully connected layers that takes a one-dimensional vector of raw bellow signal or latent variables and produces a one-dimensional vector that can be used to multiply or add to two-dimensional channels. Accordingly, the term “bellow” may refer to a surrogate signal that is a one-dimensional breathing signal (or other biological signals) generated by the model. Accordingly, the external surrogate can be any type of signal, e.g., a one-dimensional signal captured by a sensor (e.g., chest belt of height of block placed on chest) or a two-dimensional surface imaging signal. In the embodiments where the surrogate signal is high dimensional, a dimensional reduction can be performed such that principal varying low dimensional components can be used in a similar fashion as the bellows signal.

FIG. 5 illustrates a mapping block of generating a bellow signal in accordance with an embodiment. As depicted, the bellow mapping block 500 may ingest one-dimensional vectors of either raw current bellow signal or of latent vectors representing long bellow history signal. The mapping block 500 may then generate one or more vectors of “weights,” which can be used to “condition” two-dimensional or three-dimensional channels by multiplication, addition, or other methods.

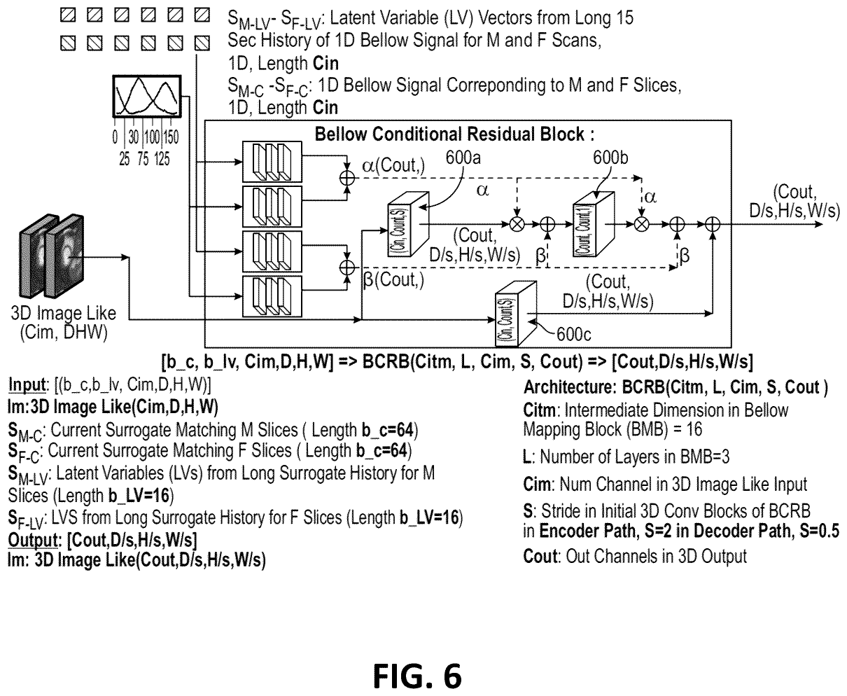

In some embodiments, a below conditional residual block may be used. Bellow conditional residual blocks may extend the concept of standard residual neural network block by incorporating multiple bellow mapping blocks, each of which can generate multiplication factor and addition offsets from the one-dimensional bellow signals and influences (e.g., via multiplication, addition) channels of convolution neural network layers that process the two-dimensional image or three-dimensional volumetric inputs. FIG. 6 depicts the concept of bellow conditional residual block.

FIG. 6 depicts an architecture of a bellow conditional residual block, in accordance with an embodiment. As in standard residual blocks, the depicted architecture may use three-dimensional convolution layers (e.g., blocks 600a-c) in direct and residual paths to obtain multiple channels of features from three-dimensional image-like input. In some embodiments, the current and long history bellow signal (in the form of latent vector representation) may influence those feature channels in the form of multiplicative and additive bias obtained via bellow mapping blocks.

### Modeling Diffeomorphic Deformation

In some embodiments, the deformation between the thin slabs (scans) may be modeled as a diffeomorphic transformation. In order to achieve this, the AI model/analytics server may utilize manifolds. As used herein, a manifold may refer to a topological space that is locally Euclidean (e.g., around every point, there is a neighborhood that is topologically the same as the open unit ball in Rn). In general, any object that is nearly “flat” on small scales may be considered a manifold. Accordingly, manifolds constitute a generalization of objects. Moreover, diffcomorphism, as used herein, may refer to a smooth, differentiable, invertible map between manifolds (e.g., between points on one manifold to points on another manifold). Diffcomorphism may also be defined as a mathematical description of a realistic deformation of a structure.

In a non-limiting example, assume that x=f (X, Y, Z); y=g (X, Y, Z); z=h (X, Y, Z) is a description of the deformation of a particular structure. To be a diffcomorphism, the functions may disallow the material being to be ripped apart (no hole formation) or to be fused into another matter. To be a diffeomorphism, the functions f, g, and h may need to be differentiable everywhere and globally invertible in the sense that there exist functions F, G, and H such that the above set of equations is solvable uniquely for XYZ in terms of xyz: X=F(x,y,z) Y=G(x,y,z) Z=H(x,y,z).

One parameterization of diffeomorphic transformation may be a stationary velocity field parameterization. Here, a diffeomorphic deformation field is determined by a smooth time-varying velocity field vₜ, t∈[0,1] via the following ODE:

$$\frac{d}{dt} {∅}_{t} = {v}_{t} o {∅}_{t}$$

with Øₜ indicating the deformation at the time point t. Ø₀ is the identity map and Ø₁ is the deformation that transforms the source image I₀ to the target image I₁. Given the velocity fields vₜ and Ø₀, the computation of Ø₁ is the numerical integration of the above equation, given as Ø₁=Ø₀+∫₀¹vₜ(Øₜ)dt.

The underlying lie group of diffeomorphisms can be parameterized by stationary velocity fields, (vₜ=V, ∀t). This one-parameter subgroup may be governed by the ODE:

$$\frac{d}{dt} {∅}_{t} = V ( {∅}_{t} )$$

For the flow of a stationary velocity vector field in the above equation, the solution of Øₜ(t) may be represented as the exponential of the velocity V given as:

Ø(t)=exp(tV)

Based on the above formulation, the AI model may model deformation as exponentiation of smoothed stationary velocity that is conditional on anatomy and change between surrogate biological signals (bellows).

### Bellow Conditional Variational Auto Encoder (CVAE)

The AI model may use a CVAE architecture. In some embodiments, the AI model may generate a CVAE architecture that intakes one or more three-dimensional slabs of slices corresponding to a “Fixed (F)” scan as input and employs as conditional input a three-dimensional slab of slices corresponding to “Moving (M)” scan, a one-dimensional raw “current” bellow signal, and a one-dimensional latent vector from bellow signal history.

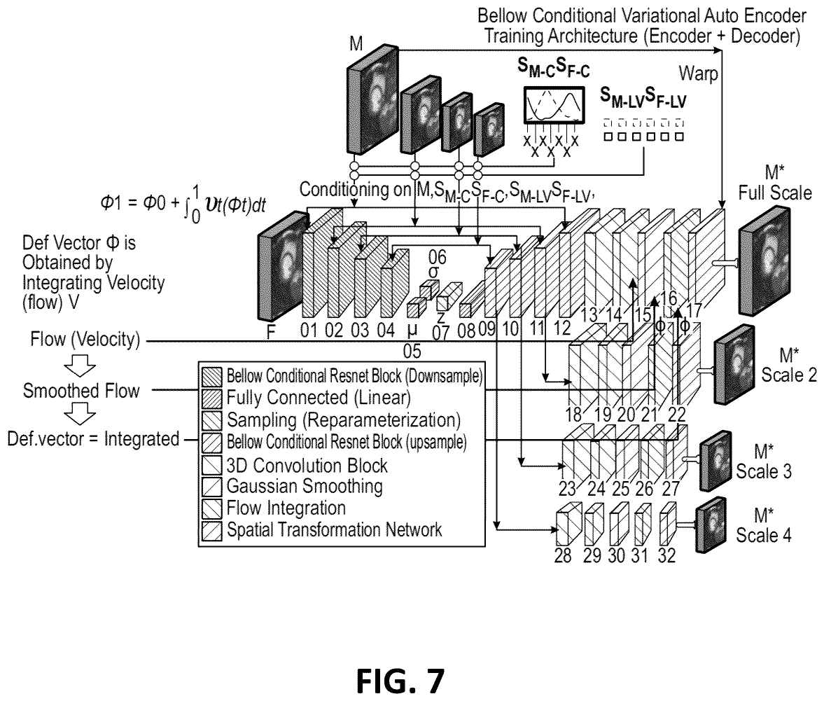

A bellow conditional variational autoencoder is depicted in FIG. 7, in accordance with an embodiment. In the depicted system architecture, three-dimensional slabs of slices (sometimes referred to as moving M volume) as well as one-dimensional breathing signal (long history and/or current) matching acquisition of both moving and fixed (F) scan may be employed as conditional input and a three-dimensional slab of slices of fixed (F) scan may be used as input.

The CVAE may be trained using traditional CVAE loss functions, which may consist of reconstruction loss between deformed image M*, F, and Kulback Liebler divergence loss between multivariate normal distributions N(μ, σ) and N(0,1) where μ, σ are generated by the encoder path of the CVAE.

Once the CVAE is trained, the decoder path of the trained model may accept thin slabs M of a reference scan, corresponding breathing signal S_{M}:<S_{M_H}, S_{M_C}> and new breathing signal (possibly during treatment) S_{F}:<S_{F_H}, S_{F_C}> as conditional input and random sampling from N(0, 1) as input to obtain deformed thin slabs M*.

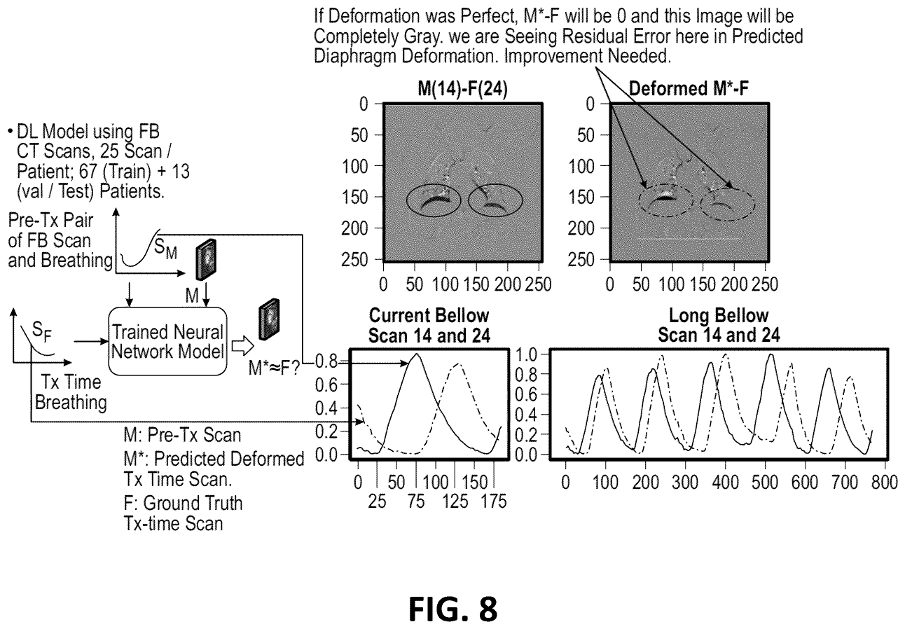

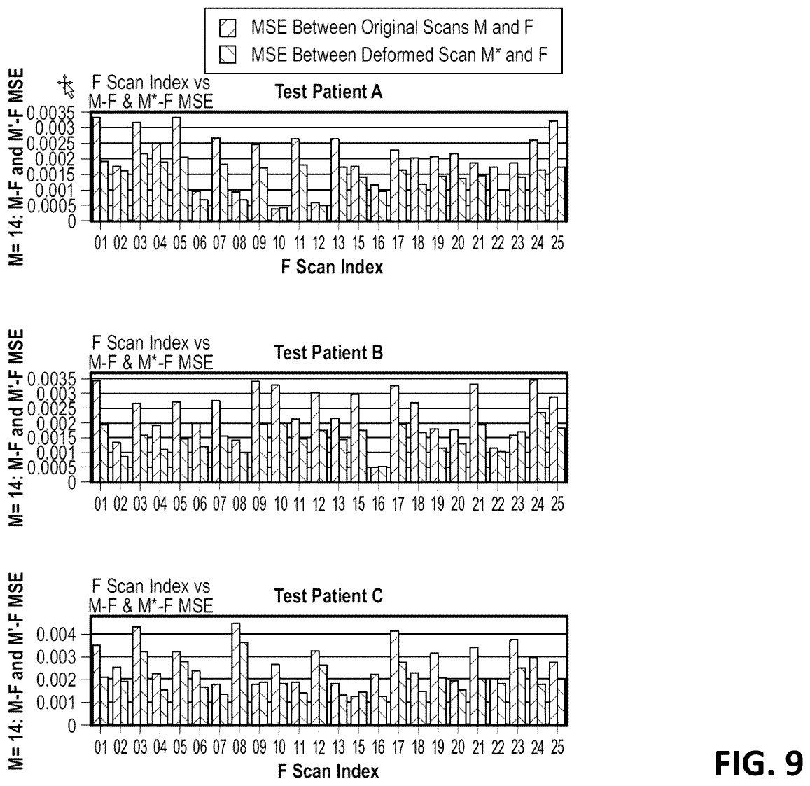

In a non-limiting example, depicted in FIGS. 8-9, the AI model may be trained using only 67 patients in a cohort of patients. Specifically, the model may be trained with 16-slice thin slabs from 25 free breathing scans per patient of the cohort of 67 patients. Then for each of the 13 test patients, each of which also has 25 free breathing scans, one of the scans (scan No. 14) may be identified as a reference scan. Then for each pair of scans (i, j), i=14, 1≤j≤25, j≠14 of a test patient the AI model may identify “matching” thin slabs (M) from scan i=14, corresponding breathing surrogate S_{M} and thin slabs (F) from scan 1≤j≤25, j≠14, corresponding breathing surrogate S_{F}.

With the trained AI model in generation mode, M, S_{M}, S_{F} may be passed as conditional input and deformed thin slab M* may be generated. Combining the deformed thin slabs M*, may result in generating a complete deformed volume prediction of scan i, which can now be compared (in terms of pixel-wise Mean Square Error (MSE)) with ground truth scan j.

FIG. 8 depicts that, for a test patient, inputting breathing signal S_{F} corresponding to scan j=24 deforms the reference scan i=14 and predicts a deformed anatomy that matches scan j=24.

FIG. 9 depicts how for three test patients, MSE error M*−F is less than M-F when M* has been generated from M conditional on the change in breathing surrogate from S_{M} to S_{F}.

The AI model discussed herein and trained using the methods and systems described herein may be a population-based model. Therefore, unlike conventional AI models, the AI model discussed herein does not require a large number of free breathing scans for a new patient. In some embodiments, a single reference free-breathing scan is enough to generate deformation to a new breathing signal. In some embodiments, more free-breathing scans may be needed; however, the AI model discussed herein generally requires fewer scans than conventionally required.

Moreover, while some conventional methods model deformation as a function of anatomy, the AI model discussed herein may model the deformation as a function of both anatomy as well as the biological signal (e.g., breathing) signal.

Some conventional methods generate a model of deformation conditional to surrogate to-dimensional images, which requires explicit generation of deformation from a pre-trained model. In contrast, the AI model discussed herein may not use any pre-trained deformation model. The AI model discussed herein may use components relating thin slabs with historical and current breathing signals, such that a volume can be predicted slice by slice by knowing the surrogate signal history until the acquisition of the slice.

The AI model discussed herein may also simulate breath-hold or 4DCT volume. In order to achieve the same, conventional methods require reference 4DCT scans at extreme inhalation and exhalation and an encoding of any 4DCT breathing phase as a number 0 and 1. In contrast, the AI model discussed herein may only need a free breathing scan, which is an improvement compared to conventional methods and modeling techniques. Moreover, the usage of free breathing scans as native data may also reduce the problem of sorting artifacts present in input 4DCT data.

While the AI model discussed herein uses free breathing scans and continuous one-dimensional surrogate breathing signals, the methods and systems discussed herein can be used to generate/train a model in a similar fashion using 4DCT scans and discrete labels if free breathing scans are not available.

The usage of latent representation for one-dimensional historical breathing signals as input also suggests a natural way of sensor fusion or usage of alternate surrogates-like sequences of surface images or two-dimensional slices. In alternative embodiments, a latent representation of various types of surrogate signals can also be obtained by building a secondary model (e.g., VAE). Then, during training (and generation) of this generative model, such diverse or alternate surrogate signals can be combined or used in the form of their latent codes in a bellow conditional residual block.

While the embodiments discussed herein describe using a CVAE architecture, in alternative embodiments, this architecture may be replaced by various generative models, such as Generative Adversarial Networks (GAN) or Normalizing Flow while using the structure like Bellow Conditional Resnet Block as building blocks.

### Execution and Implementation of the AI Model

Referring back to FIG. 2, at step 202, the analytics server may receive, via an electronic sensor, biological signal data of a patient. The analytics server may receive respiratory data of a patient from an electronic sensor, such as a chest belt. Alternatively, the patient's electrocardiogram or Kv imaging can be used as the biological signal.

In embodiments where breathing is used as the biological signal, the analytics server may be in communication with one or more sensors configured to monitor a patient's movements. The electronic sensor may identify the respiratory rate of the patient by counting the number of breaths and how many times the patient's chest rises. In one example, the electronic sensor may be a wearable (e.g., chest strap or a patch over the chest) respiratory monitoring system that monitors the respiratory patterns of a patient. The electronic sensor may detect small changes in a patient's breathing pattern, chest position, tidal volume, and/or other vital signs.

In another example, a fiber-optic breath rate sensor can be used for monitoring the patient. In yet another example, various 3D surfacing methods may be used to determine how the patient is breathing. Additionally, the analytics server may retrieve one or more medical images (e.g., CT or 4DCT) of the patient.

At step 204, the analytics server may receive a medical image of the patient depicting a planning target volume and at least one organ at risk of the patient, wherein the medical image received corresponds to the patient in a pre-treatment condition. As discussed herein, for instance, with respect to FIG. 3, the analytics server may receive pre-treatment (“free” and unobstructed) medical images (3D volumes) of the patient's internal structures. In a non-limiting example, the patient may be asked to breathe freely while an electronic sensor is monitoring the patient's respiratory data and an imaging apparatus is collecting medical images of the patient in free-breathing mode. If other biological signals are used, then pre-treatment medical images (when the patient is relaxed) can be used instead of the “free breathing” medical images.

The medical images may depict the patient's PTV and OAR and may indicate how they move in relation to the pre-treatment (free breathing) biological signal.

At 204, the analytics server may execute an artificial intelligence model using the biological signal data and the medical image to predict deformation data for at least one of the at least one organ at risk or the planning target volume of the patient, wherein the artificial intelligence model is trained in accordance with a training dataset comprising a set of participants, their corresponding respiratory data, their medical images, and their corresponding deformation data.

As used herein, deformation data may refer to any data predicted by the AI model. Non-limiting examples of deformation data may include any data (e.g., deformation vectors, numbers, and simulated medical images) that convey how one or more internal structures would move or deform at a given time.

The analytics server may execute the AI model discussed herein using the data received in step 202. Additionally, the analytics server may receive an initial medical image of the patient (step 204). The AI model may be trained in accordance with the methods and systems discussed herein. The AI model may ingest the patient's pre-treatment medical images. Using this data, the AI model may personalize itself to the patient, even though the AI model is trained using other patient's data.

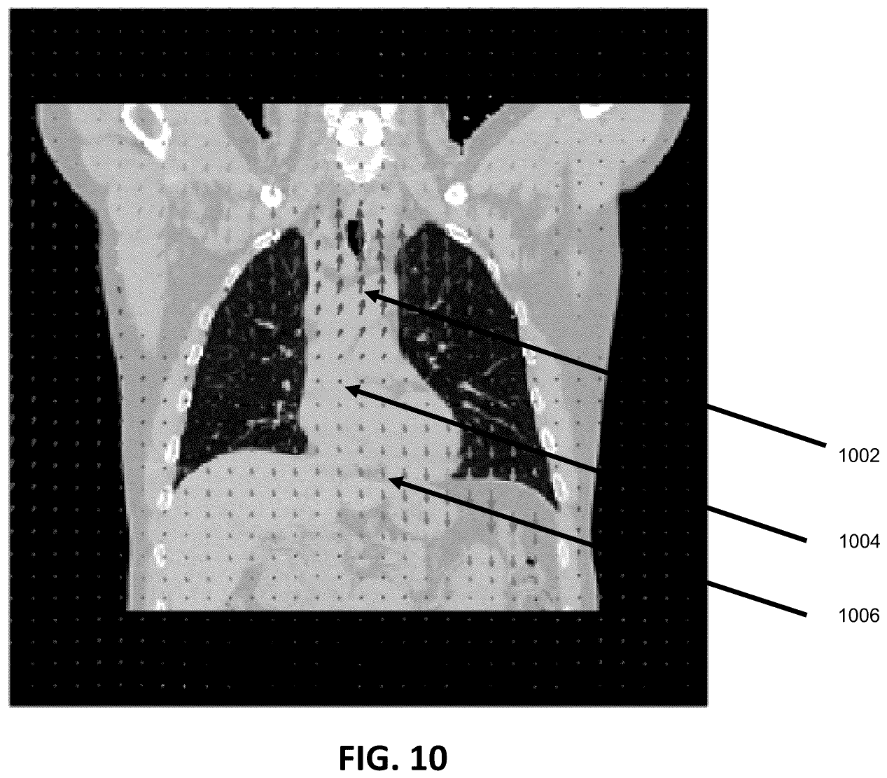

The AI model may predict deformation data associated with one or more organs or internal structures of the patient. Specifically, the AI model may predict deformation vectors indicating how each point within a medical image of the patient will move/deform. The deformation vectors may indicate the distance and direction in which each point within the medical image will move. For instance, as depicted in the deformation vectors depicted in FIG. 10. For instance, a vector 1002 indicates that its corresponding point within the medical image will move upwards (e.g., by 1 millimeter), and a vector 1004 indicates that its corresponding location will not move. In contrast, a vector 1006 indicates that its corresponding location will move downwards (e.g., 0.5 millimeters). Using the deformation vectors, the analytics server may predict the location and orientation of one or more internal strictures of the patient.

At step 208, the analytics server may output the data predicted by the AI model (deformation data). The analytics server may output the deformation data in multiple ways. In one embodiment, the analytics server may output the deformation vectors. For instance, a GUI accessed by a medical professional may display an image like the one depicted in FIG. 10, where different deformation vectors and their corresponding magnitude and direction are depicted.

In another example, the analytics server may use the AI model to generate a moving or fixed medical image that depicts how the patient's internal structure would move/deform. For instance, a GUI accessed by a medical professional may display a projected 4DCT of the patient that depicts how the patient's internal structures are going to move/deform.

In another example, the analytics server may revise one or more attributes of the patient's radiotherapy treatment using the data predicted by the AI model. For instance, the analytics server may revise an attribute of a multi-leaf collimator (MLC), move the couch, or pause the beam, or a combination of any of these examples. Specifically, in conjunction with one or more other software solutions, the analytics server may revise an opening of the MLC, such that radiation dissemination is directed towards the projected location of a PTV (e.g., projected using the Al model). In this way, the analytics server provides a dynamic MLC correction method where the MLC opening can be revised in real-time or near real-time.

Effectively, the analytics server may enable “gating” of the beam to match the motion of the patient's tumor. Because the analytics server can predict/estimate the tumor location, the analytics server may control one or more attributes of the radiotherapy machine. For instance, the analytics server may control (e.g., review and revise) the MLC opening, timing, and/or the dose rate.

In another example, the analytics server may transmit the data predicted via the AI model to a downstream software solution. For instance, the results of the execution of the AI model can be transmitted to a dose calculation software solution. In another example, the analytics server may transmit the deformation data to a downstream tissue-tracking application.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
receiving, by a processor via an electronic sensor, biological signal data of a patient;
receiving, by the processor, a medical image of the patient depicting a planning target volume and at least one organ at risk of the patient, wherein the medical image received corresponds to the patient in a pre-treatment condition;
executing, by the processor, an artificial intelligence model using the biological signal data and the medical image to predict deformation data for at least one of the at least one organ at risk or the planning target volume of the patient,
wherein the artificial intelligence model is trained in accordance with a training dataset comprising a set of participants, their corresponding respiratory data, their medical images, and their corresponding deformation data; and
outputting, by the processor, the deformation data.

2. The method of claim 1, wherein the medical image is a free-breathing image of the patient.

3. The method of claim 1, wherein the artificial intelligence model is configured to use a moving or fixed portion of the planning target volume or the at least one organ at risk within the medical image to predict the deformation data.

4. The method of claim 1, wherein the biological signal data received from the electronic sensor is at least one of a chest position, chest movement, or respiratory cycle data of the patient.

5. The method of claim 1, wherein the deformation data corresponds to a movement, change in shape, or a position of at least one of the planning target volume or the at least one organ at risk of the patient.

6. The method of claim 1, further comprising:
adjusting, by the processor, at least one attribute of a radiotherapy machine in accordance with the deformation data.

7. The method of claim 1, wherein the electronic sensor is a wearable respiratory sensor or an optical respiratory sensor.

8. The method of claim 1, wherein outputting the deformation data corresponds to a simulated medical image depicting an anatomical region of the patient.

9. The method of claim 1, wherein outputting the deformation data corresponds to transmitting the deformation data to a dose calculation software solution or a tissue tracking software solution.

10. A computer system comprising:
a server comprising a processor and a non-transitory computer-readable medium containing instructions that when executed by the processor causes the processor to perform operations comprising:
receiving, via an electronic sensor, biological signal data of a patient;
receiving a medical image of the patient depicting a planning target volume and at least one organ at risk of the patient, wherein the medical image received corresponds to the patient in a pre-treatment condition;
executing an artificial intelligence model using the biological signal data and the medical image to predict deformation data for at least one of the at least one organ at risk or the planning target volume of the patient,
wherein the artificial intelligence model is trained in accordance with a training dataset comprising a set of participants, their corresponding respiratory data, their medical images, and their corresponding deformation data; and
outputting the deformation data.

11. The computer system of claim 10, wherein the medical image is a free-breathing image of the patient.

12. The computer system of claim 10, wherein the artificial intelligence model is configured to use a moving or fixed portion of the planning target volume or the at least one organ at risk within the medical image to predict the deformation data.

13. The computer system of claim 10, wherein the biological signal data received from the electronic sensor is at least one of a chest position, chest movement, or respiratory cycle data of the patient.

14. The computer system of claim 10, wherein the deformation data corresponds to a movement, change in shape, or a position of at least one of the planning target volume or the at least one organ at risk of the patient.

15. The computer system of claim 10, wherein the instructions further cause the processor to:
adjust at least one attribute of a radiotherapy machine in accordance with the deformation data.

16. The computer system of claim 10, wherein the electronic sensor is a wearable respiratory sensor or an optical respiratory sensor.

17. The computer system of claim 10, wherein outputting the deformation data corresponds to a simulated medical image depicting an anatomical region of the patient.

18. The computer system of claim 10, wherein outputting the deformation data corresponds to transmitting the deformation data to a dose calculation software solution or a tissue tracking software solution.

19. A system comprising:
a radiotherapy machine;
a data repository configured to store an artificial intelligence model;
a processor configured to:
receive, via an electronic sensor, biological signal data of a patient;
receive a medical image of the patient depicting a planning target volume and at least one organ at risk of the patient, wherein the medical image received corresponds to the patient in a pre-treatment condition;
execute the artificial intelligence model using the biological signal data and the medical image to predict deformation data for at least one of the at least one organ at risk or the planning target volume of the patient,
wherein the artificial intelligence model is trained in accordance with a training dataset comprising a set of participants, their corresponding respiratory data, their medical images, and their corresponding deformation data; and
output the deformation data.

20. The system of claim 19, wherein the medical image is a free-breathing image of the patient.
